(19) United States
(12) Patent Application Publication
(10) Pub. No.: US 2007/0112414 A1
(43) Pub. Date: May 17, 2007

(54) System and method for local delivery of antithrombotics

(75) Inventors: Theodore L. Parker, Danville, CA (US); Frank Litvack, Los Angeles, CA (US); Micheline Lisa Markey, Atherton, CA (US)

Correspondence Address: CINDY A. LYNCH;CONOR MEDSYSTEMS, INC., 1003 HAMILTON COURT, MENLO PARK, CA, 94025, US

(73) Assignee: Conor Medsystems, Inc., Menlo Park, CA (US)

(21) Appl. No.: 11/516,054

(22) Filed: Sep. 5, 2006

Related U.S. Application Data
(60) Provisional application No. 60/715,818, filed on Sep. 8, 2005.

(51) Int. Cl. A61F 2/06

(52) U.S. Cl. 623/1.15; 623/1.42

(57) ABSTRACT

Dipyridamole is an antithrombotic agent which also promotes the growth of endothelial cells. An endothelial cell lining within a stent is necessary for complete healing on the interior of the stent. A dual drug dipyridamole stent includes a first drug formulation of dipyridamole and polymer arranged in a first set of holes in the stent for primarily luminal delivery and a second drug formulation of an antirestenotic agent and polymer arranged in a second set of holes in the stent for primarily mural delivery. The delivery of dipyridamole luminally into the blood stream can involve a two phase release with the first phase being a burst to prevent initial clotting or thrombus formation followed by a second phase with a much slower and more sustained release to reduce thrombogenicity and promote the growth of the endothelial cell lining.

### CROSS REFERENCE TO RELATED APPLICATIONS

This application claims priority to U.S. Provisional Patent Application Ser. No. 60/715,818, filed Sep. 8, 2005 the entire contents of which are incorporated herein by reference.

### BACKGROUND

A variety of diseases, including atherosclerosis, cause a narrowing of the lumen of blood vessels. This is of particular concern when there is a formation of plaque in a coronary artery because the chronic condition of ischemia, causing angina, or the acute formation of a thrombus or clot that blocks the artery at the position of the plaque can cause myocardial infarction. Cardiovascular stents are deployed directly into vascular tissue, such as arteries and veins, at the position of the plaque, to open the stenosed vessel.

Healthy blood vessels are lined with endothelial cells, which present a non-thrombogenic surface. However, when blood comes into contact with foreign surfaces (such as a stent), collagen from the vessel wall, or smooth muscle cells if the endothelium is breached, as in vulnerable plaque, platelets are activated and blood coagulation is initiated. Therefore, there is the possibility that a clot or thrombus may form in the stent soon after blood flow is re-established.

Since restenosis of the artery following the stent placement is also a problem, stents that elute antirestenotic agents have been developed. However, it is still beneficial that an antithrombotic also be eluted for some sustained period following the immediate, or burst, release at the time of stent placement.

Thus, a stent that combines an antirestenotic agent and an antithrombotic agent has utility over either a standard antirestenotic stent or an antithrombotic eluting or coated stent.

### DETAILED DESCRIPTION

Definitions

The terms “agent” or “beneficial agent” as used herein are intended to have the broadest possible interpretation and are used to include any therapeutic agent or drug, as well as inactive agents such as barrier layers, carrier layers, therapeutic layers, or protective layers.

The terms “drug” and “therapeutic agent” are used interchangeably to refer to any therapeutically active substance that is delivered to a bodily lumen of a living being to produce a desired, usually beneficial, effect. Beneficial agents may include one or more drug or therapeutic agent.

The terms “openings” and “holes” includes both through openings and recesses.

The term “polymer” refers to molecules formed from the chemical union of two or more repeating units, called monomers. Accordingly, included within the term “polymer” may be, for example, dimers, trimers and oligomers. The polymer may be synthetic, naturally-occurring or semisynthetic. In preferred form, the term “polymer” refers to molecules which typically have a Mw greater than about 3000 and preferably greater than about 10,000 and a Mw that is less than about 10 million, preferably less than about a million and more preferably less than about 200,000. Examples of polymers include but are not limited to, poly-O-hydroxy acid esters such as, polylactic acid (PLLA or DLPLA), polyglycolic acid, polylactic-co-glycolic acid (PLGA), polylactic acid-co-caprolactone; poly (block-ethylene oxide-block-lactide-co-glycolide) polymers (PEO-block-PLGA and PEO-block-PLGA-block-PEO); polyethylene glycol and polyethylene oxide, poly (block-ethylene oxide-block-propylene oxide-block-ethylene oxide); polyvinyl pyrrolidone; polyorthoesters; polysaccharides and polysaccharide derivatives such as polyhyaluronic acid, poly (glucose), polyalginic acid, chitin, chitosan, chitosan derivatives, cellulose, methyl cellulose, hydroxyethylcellulose, hydroxypropylcellulose, carboxymethylcellulose, cyclodextrins and substituted cyclodextrins, such as beta-cyclodextrin sulfobutyl ethers; polypeptides and proteins, such as polylysine, polyglutamic acid, albumin; polyanhydrides; polyhydroxy alkonoates such as polyhydroxy valerate, polyhydroxy butyrate, and the like.

The term “primarily” with respect to directional delivery, refers to an amount greater than 50% of the total amount of therapeutic agent provided to a blood vessel is provided in the primary direction.

Dipyridamole is an antithrombotic agent that has shown utility in systemic administration. Dipyridamole is a particularly attractive drug to use to reduce the thrombogenicity of the surface of the stent and inhibit platelet activation and aggregation because it also promotes the growth of an endothelial cell lining within the stent, which in is necessary for complete healing on the interior of the stent. A complete endothelial cell lining is needed to separate the stent from direct blood contact and reduce late occurrences of stent thrombus or clotting.

Dipyridamole (Persantine) is a vasodilator that in combination with aspirin reduces thrombosis in patients with thrombotic diseases, such as a previous stroke. Dipyridamole is delivered systemically by an oral capsule and interferes with platelet function by increasing the cellular concentration of adenosine 3′-5′-monophosphate. Dipyridamole reduces platelet adhesion as well as aggregation, probably by inhibiting phosphodiesterase and so raising platelet cyclic AMP levels. Dipyridamole acts as an antithrombotic and also acts as a vasodilator which can help to maintain the open lumen of the blood vessel.

Dipyridamole can be delivered from a stent in a luminal direction while an antirestenotic agent is delivered morally to reduce restenosis. One method of luminal release of dipyridamole is a two phase release with the first phase being a burst to prevent initial clotting or thrombus formation followed by a second phase with a much slower and more sustained release to reduce thrombogenicity and promote the growth of the endothelial cell lining over a time period to achieve a substantially complete endothelial cell lining.

In one example dipyridamole is mixed with PLGA or other bioresorbable polymer in a solvent and is deposited in through holes in a stent. The solvent is then evaporated and the procedure is repeated to deposit a desired dose of dipyridamole. Methods and systems for depositing polymers and drugs within holes in stents are described further in WO 2004/026182 which is incorporated herein by reference. To provide primarily luminal delivery, the dipyridamole/PLGA deposit is covered with a polymer deposit which acts as a cap and substantially prevents mural delivery. The polymer cap can be formed of a slower degrading polymer than the polymer used with the dipyridamole. Preferably, at least 80% of the dipyridamole is delivered luminally.

The dose of dipyridamole delivered can vary between about 5 and about 200 micrograms. In one example, the dose is about 20 to about 100 micrograms delivered primarily luminally.

Due to the possibility that a clot may form in the stent soon after blood flow is re-established it is beneficial if the stent can begin eluting an antithrombotic agent immediately upon implantation. For example, dipyridamole can be delivered to the blood stream in a bolus or burst of about 1 to about 50 micrograms, preferably about 20-50 micrograms in the first few hour or day after implantation. This burst release will provide the antithrombotic drug into the bloodstream to reduce or prevent clot formation. The first phase can be achieved by the combination of dipyridamole with a first or fast release polymer.

After the first about 24 hours following stent implantation an inner layer of fibrin forms over the stent and the stent is substantially encased. Thus, following the burst delivery of antithrombotic, the remaining antithrombotic is delivered into the fibrin for delivery to the circulation or remains in the fibrin to provide an antithrombotic surface. The antithrombotic can be continuously released at a low level for several weeks or months to continue to provide an antithrombotic surface. This second phase can be achieved by the combination of dipyridamole with a second or slower release polymer placed on top (in the mural direction) of the first phase.

Once a complete endothelial cell lining has formed within the stent, which generally occurs between about 20 and 60 days, delivery of the antithrombotic agent can end.

As described above, a stent that combines an antirestenotic agent and an antithrombotic agent has utility over either a standard antirestenotic stent or an antithrombotic eluting or coated stent.

There are a number of antirestenotic agents which can be used in combination with dipyridamole to achieve the combined effects of antirestenotics and antithrombotics. These antirestenotic agents include, without limitation, the antiproliferatives sirolimus, rapamycin, and other limus drugs (also called immunosuppressants), paclitaxel, actinomycin D, cyclosporin, and Zotarolimus.

Pimecrolimus is an immunosuppressant that has been shown to have antirestenotic properties. Thus, a stent that combines the sustained local luminal release of the antithrombotic dipyridamole and the sustained mural release of the inmmunosuppressant, and antirestentotic, Pimecrolimus provides protection against restenosis and also provides protection against acute and longer term thrombus formation within the stent that could result in an AMI.

When more than one agents, such as an antithrombin and an antirestenotic are incorporated in a stent or other implantable medical device they may be incorporated by use of holes, reservoirs, coatings or other known means. When holes or reservoirs are used, more than one agent can be placed in one hole for directional delivery in the same or different directions. Alternatively, the different agents can be incorporated in different holes or reservoirs to achieve release characteristics tailored to the treatment desired. Further, the same agent can be incorporated in more than one formulation or polymer in the same or different holes to achieve a desired release, such as a burst formulation and a sustained release formulation of the same agent in different holes interspursed along the stent.

Although the invention has been described using the antithrombotic dipyridamole as a primary example, other antithrombins can also be used in place of dipyridamole. Antithrombins include, without limitation, heparin, aspirin, sulfinpyrazone, ticlopidine, ABCIXIMAB, eptifibatide, tirofiban HCL, coumarines, plasminogen, [symbol] -antiplasmin, streptokinase, urokinase, bivalirudin, tissue plasminogen activator (t-PA), hirudins, hirulogs, argatroban, hydroxychloroquin, BL-3459, pyridinolcarbamate, Plavix, and Angiomax.

Dipyridamole may also be combined with an antirestenotic and with another antithrombotic, such as aspirin.

Although the invention has been described as incorporating the agents described above into the stent in holes in the stent, one or more of the agents can be incorporated in the stent by one or more of the other known means, such as coating, affixing threads, microspheres, or sleeves.

While the invention has been described in detail with reference to the preferred embodiments thereof, it will be apparent to one skilled in the art that various changes and modifications can be made and equivalents employed, without departing from the present invention.

What is claimed is:

1. A method of delivering dipyridamole locally within a body lumen, the method comprising:
providing a substantially cylindrical implantable medical device having dipyridamole affixed thereto for local delivery within a patient;
implanting the substantially cylindrical implantable medical device into a body lumen; and
directionally delivering dipyridamole primarily into the body lumen, without delivering substantial dipyridamole directly to a wall of the body lumen.

2. The method of claim 1, wherein the substantially cylindrical implantable medical device is a stent.

3. The method of claim 1, wherein the substantially cylindrical implantable medical device is a vascular stent.

4. The method of claim 1, wherein the body lumen is a blood vessel.

5. The method of claim 1, wherein the dipyridamole is delivered in a two phase release with a first burst phase of rapid release over about 1 to 24 hours and a second sustained release phase over at least 10 days.

6. The method of claim 1, wherein the substantially cylindrical implantable medical device includes an antirestenotic agent, and wherein the antirestenotic agent is directionally delivered primarily to a wall of the body lumen.

7. The method of claim 6, wherein the antirestenotic agent is pimecrolimus.

8. The method of claim 6, wherein the antirestenotic agent is paclitaxel.

9. The method of claim 6, wherein the antirestenotic agent is a limus.

10. The method of claim 1, wherein the substantially cylindrical implantable medical device includes aspirin.

11. A stent comprising:
a plurality of stent struts having holes formed therein for receiving beneficial agents for directional delivery;
a first drug formulation of dipyridamole and polymer arranged in a first plurality of holes in the stent for primarily luminal delivery; and
a second drug formulation of an antirestenotic agent and polymer arranged in a second plurality of holes in the stent for primarily mural delivery.

12. The stent of claim 11, wherein the first plurality of holes and the second plurality of holes are different holes in the stent which are interspersed.

13. The stent of claim 11, wherein the first plurality of holes and the second plurality of holes are the same holes in the stent.

14. The stent of claim 11, wherein the antirestenotic agent is Pimecrolimus.

15. The stent of claim 11, wherein the antirestenotic agent is paclitaxel.

16. The stent of claim 11, wherein the antirestenotic agent is a limus.

17. The stent of claim 11, wherein the stent device includes aspirin.

18. A stent comprising:
a plurality of struts having holes formed therein for receiving beneficial agents for directional delivery;
a first drug formulation including a bioresorbable polymer and at least one drug from the group consisting of dipyridamole, heparin, aspirin, sulfinpyrazone, ticlopidine, ABCIXIMAB, eptifibatide, tirofiban HCL, coumarines, plasminogen, α₂-antiplasmin, streptokinase, urokinase, bivalirudin, tissue plasminogen activator (t-PA), hirudins, hirulogs, argatroban, hydroxychloroquin, BL-3459, pyridinolcarbamate, Plavix, and Angiomax arranged in a first plurality of the holes in the stent struts for primarily luminal delivery; and
a second drug formulation of a bioresorbable polymer and an antirestenotic agent arranged in a second plurality of the holes in the stent struts for primarily mural delivery.

19. The stent of claim 18, wherein the first plurality of holes and the second plurality of holes are different holes in the stent which are interspersed.

20. The stent of claim 18, wherein the first plurality of holes and the second plurality of holes are the same holes in the stent.

21. The stent of claim 18, wherein the antirestenotic agent is Pimecrolimus.

22. The stent of claim 18, wherein the at least one drug is arranged in the first plurality of holes for a two phase release with a first burst phase of rapid release over about 1 to 24 hours and a second sustained release phase over at least 10 days.

23. The stent of claim 22, wherein the two phase release is achieved by the at least one drug having a first formulation in a portion of the first plurality of holes and a second formulation in a second portion of the first plurality of holes.
